(19) United States
(12) Patent Application Publication
(10) Pub. No.: US 2011/0150946 A1
(43) Pub. Date: Jun. 23, 2011

(54) Transdermal Delivery of Apomorphine Using Microneedles

(76) Inventor: Abeer M. Al-Ghananeem, Lexington, KY (US)

(21) Appl. No.: 13/060,118

(22) PCT Filed: Aug. 21, 2009
(86) PCT No.: PCT/US09/54604

§ 371 Date: Feb. 22, 2011

Related U.S. Application Data
(60) Provisional application No. 61/090,989, filed on Aug. 22, 2008.

(51) Int. Cl. A61K 9/00; A61K 31/473; A61P 25/16; A61P 25/18; A61P 25/24; A61P 25/30; A61P 15/10

(52) U.S. Cl. 424/400; 514/284

(57) ABSTRACT

The present invention relates generally to methods for transdermal delivery of a therapeutically effective amount of apomorphine using microneedles. The invention also provides methods for treatment of erectile dysfunction and Parkinson's disease using apomorphine hydrochloride or any pharmaceutically acceptable salt, and/or apomorphine prodrugs to the microneedle-treated site.

### CROSS-REFERENCE TO RELATED APPLICATIONS

This application claims priority to U.S. Provisional Patent Application Ser. No. 61/090,989, which was filed on Aug. 22, 2008, the entirety of which is incorporated herein by reference for all purposes.

### FIELD OF THE INVENTION

The present invention relates generally to methods for transdermal delivery of a therapeutically effective amount of apomorphine using microneedles. The invention also provides methods for treatment of erectile dysfunction and Parkinson's disease using apomorphine hydrochloride or any pharmaceutically acceptable salt, and/or apomorphine prodrugs to the microneedle-treated site.

### BACKGROUND OF THE INVENTION

Apomorphine (APO) is a dopamine D₁ and D₂ receptor agonist, which has been used in the treatment of a variety of medical conditions, including Parkinson's disease. The dopaminergic properties of APO were first recognized in the 1960s, when it was successfully used to suppress refractory motor oscillations in Parkinson's disease. Furthermore, through activation of dopamine D₁ and D₂ postsynaptic receptors which are mainly located in the paraventricular nucleus of the hypothalamus, apomorphine has been shown to be effective in eliciting penile erection in both rat and human models.

Erectile dysfunction (ED) is a common medical condition that affects the sexual life of millions of men worldwide. Compared with those phosphodiesterase type 5 inhibitors such as sildenafil, tadalafil and vardenafil, sublingual apomorphine (Uprima™) is a centrally acting agent licensed for the treatment of ED in most European and South American countries. However, the duration of action is limited by the sublingual route immediate release profile.

Because of severe first-pass metabolism, oral delivery of apomorphine appears to be virtually ineffective. On the other hand, the bioavailability of sublingual apomorphine is estimated to be 16-18%, as it is rapidly absorbed and can avoid the first-pass metabolism. However, as a marked increase in the prevalence of nausea (14.1% of incidence) was evident in the group treated with sublingual apomorphine 4 mg per patient, the patient should always start at a lower dose followed by a dose-optimization schedule.

Nasal delivery of apomorphine was studied also due to the attractive properties of this delivery route because it offers some advantages including rapid absorption, avoidance of hepatic first-pass metabolism, and the preferential drug delivery to brain. However, some side effects were reported on apomorphine clinical trials such as nausea and irritation which were considered as a limiting factor.

The brief duration of the apomorphine clinical action can be explained by its rapid clearance. For that reason, the transdermal delivery of apomorphine may be a promising strategy to provide a controlled and sustained release of apomorphine for treating Parkinson's disease and ED.

The main challenge in transdermal drug delivery is providing sufficient drug penetration across the skin. A large majority of drugs, including apomorphine, are unable to cross the skin at therapeutic rates due to the barrier imposed by the skin's outer stratum corneum layer.

Skin permeability can be increased through the use of chemical enhancers, electrical enhancers via ultrasonic enhancers, iontophoresis, and a variety of other approaches. Although these enhancement technologies are still under active investigation, delivering macromolecules into the skin remains a significant challenge. Furthermore, tingling and itching were reported among side effects during a pilot study to deliver apomorphine transdermally with permeation enhancers and iontophoresis.

An alternative approach to increase transdermal transport involves using arrays of microscopic needles (or “microneedles”) to pierce the skin, thus creating micrometer-scale transport pathways.

The present invention provides methods for transdermal delivery of a therapeutically effective amount of apomorphine Hydrochloride using microneedles. The invention also provides methods for treatment of erectile dysfunction (ED) and Parkinson's disease by inserting microneedles, followed by applying the apomorphine hydrochloride or any pharmaceutically acceptable salt, and/or apomorphine prodrugs to the microneedle-treated site.

### BRIEF SUMMARY OF THE INVENTION

The present invention overcomes the problems and disadvantages associated with current dosage forms of apomorphine by delivering the therapeutic agent transdermally through a patch-needle hybrid (Microneedle) delivery system.

It is known that the use of salts and ionized drugs is not typically optimal for standard passive transdermal dosage forms where the main route of diffusion is through the lipid bilayers of the stratum corneum. However, transport across skin treated with microneedles occurs through aqueous channels. Microneedles provide a minimally invasive means to transport molecules into the skin, as the channels they create are extremely small on a clinical level. However, such channels usually dramatically increase skin permeability.

The invention also provides methods for treatment of erectile dysfunction and Parkinson's disease comprising apomorphine hydrochloride or any pharmaceutically acceptable salt, and/or apomorphine prodrugs to the microneedle-treated site.

The present invention provides a pharmaceutical composition comprising apomorphine or a pharmaceutically acceptable salt, and/or prodrugs thereof and a pharmaceutically acceptable carrier, wherein the apomorphine or pharmaceutically acceptable salt thereof is provided in a form suitable for transdermal administration through microneedles.

These and other embodiments of the invention are described herein below or are evident to persons of ordinary skill in the art based on the following disclosures.

The above summary of the present invention is not intended to describe each embodiment or every implementation of the present invention. Advantages and attainments, together with a more complete understanding of the invention, will become apparent and appreciated by referring to the following detailed description and claims taken in conjunction with the accompanying drawings.

### BRIEF DESCRIPTION OF THE DRAWINGS

This invention, as defined in the claims, can be better understood with reference to the following drawings:

FIG. 1 is a graph depicting the in vitro release profile of apomorphine from a gel formulation through porcine skin (▴) and porcine skin pretreated with 150 micron microneedles (▪).

In the following description of the illustrated embodiments, references are made to the accompanying drawings, which form a part hereof, and in which is shown by way of illustration various embodiments in which the invention may be practiced. It is to be understood that other embodiments may be utilized and structural and functional changes may be made without departing from the scope of the present invention.

### DETAILED DESCRIPTION OF THE INVENTION

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All references, publications, patents, patent applications, and commercial materials mentioned herein are incorporated herein by reference for all purposes including for describing and disclosing the methodologies which are reported in the publications which might be used in connection with the invention. Nothing herein is to be construed as an admission that the invention is not entitled to antedate such disclosure by virtue of prior invention.

In order to provide a clear and consistent understanding of the specification and claims, including the scope to be given such terms, the following definitions are provided:

The term “administration” of the pharmaceutically active compounds and the pharmaceutical compositions defined herein includes oral and transdermal application.

By “compatible” herein is meant that the components of the compositions which comprise the present invention are capable of being mixed without interacting in a manner which would substantially decrease the efficacy of the pharmaceutically active compound under ordinary use conditions.

The terms “effective amount” or “pharmaceutically effective amount” refer to a nontoxic but sufficient amount of the agent to provide the desired biological result. That result can be reduction and/or alleviation of the signs, symptoms, or causes of a disease, such as the treatment of ED, Alzheimer's, and other therapeutic indications related to apomorphine. An appropriate “effective” amount in any individual case may be determined by one of ordinary skill in the art using routine experimentation.

As used herein, the term “excipient” means the substances used to formulate active pharmaceutical ingredients (API) into pharmaceutical formulations; in a preferred embodiment, an excipient does not lower or interfere with the primary therapeutic effect of the API. Preferably, an excipient is therapeutically inert. The term “excipient” encompasses carriers, diluents, vehicles, solubilizers, stabilizers, bulking agents, acidic or basic pH-adjusting agents and binders. Excipients can also be those substances present in a pharmaceutical formulation as an indirect or unintended result of the manufacturing process. Preferably, excipients are approved for or considered to be safe for human and animal administration, i.e., GRAS substances (generally regarded as safe). GRAS substances are listed by the Food and Drug administration in the Code of Federal Regulations (CFR) at 21 CFR 182 and 21 CFR 184, incorporated herein by reference.

As used herein, the terms “formulate” refers to the preparation of a drug, e.g., apomorphine, in a form suitable for administration to a mammalian patient, preferably a human. Thus, “formulation” can include the addition of pharmaceutically acceptable excipients, diluents, or carriers and pH adjusting agents.

The term “permeation enhancer” or “penetration enhancer” as used herein refers to an agent that improves the rate of transport of a pharmacologically active agent (e.g., apomorphine) across the transdermal tissues. Typically a penetration enhancer increases the permeability of skin to a pharmacologically active agent. Penetration enhancers, for example, enhance the rate at which the pharmacologically active agent permeates through membranes and enters the bloodstream. Enhanced permeation effected through the use of penetration enhancers can be observed, for example, by measuring the flux of the pharmacologically active agent across animal or human membranes as described in the Examples herein below. An “effective” amount of a permeation enhancer as used herein means an amount that will provide a desired enhancement in skin permeability to provide, for example, the desired depth of penetration of a selected compound, rate of administration of the compound, and amount of compound delivered.

By “pharmaceutically acceptable” or “pharmacologically acceptable” is meant a material which is not biologically or otherwise undesirable, i.e., the material may be administered to an individual without causing any undesirable biological effects or interacting in a deleterious manner with any of the components of the composition in which it is contained.

As used herein, a “pharmaceutically acceptable carrier” or “vehicle” is preferably refer to carrier materials suitable for transdermal drug administration and include any such materials known in the art, such as any liquid, gel solvent, liquid diluent, solubilizer, or the like, which is non-toxic, and which does not interact with other components of the composition in a deleterious manner. Examples of suitable carriers for use herein include water, silicone, liquid sugars, waxes, oils, petroleum jelly, and a variety of other materials. The term “carrier” or “vehicle” can also refer to crystallization inhibitors, or other types of additives useful for facilitating transdermal drug delivery. In addition, the formulation may contain additives such as thickening or gelling agents, emulsifiers, wetting agents, buffers, stabilizers, and preservatives such as antioxidants.

The term “pharmaceutical composition” as used herein shall mean a composition that is made under conditions such that it is suitable for administration to humans, e.g., it is made under current good manufacturing practice (cGMP) conditions and contains pharmaceutically acceptable excipients, e.g., without limitation, stabilizers, pH adjusting agents, bulking agents, buffers, carriers, diluents, vehicles, solubilizers, and binders.

As used herein, the term “subject” encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. The term does not denote a particular age or sex.

As used herein, the terms “treating” or “treatment” of a disease include preventing the disease, i.e. preventing clinical symptoms of the disease in a subject that may be exposed to, or predisposed to, the disease, but does not yet experience or display symptoms of the disease; inhibiting the disease, i.e., arresting the development of the disease or its clinical symptoms, such as by suppressing or relieving the disease, i.e., causing regression of the disease or its clinical symptoms.

General

This invention relates to a transdermal pharmaceutical formulations and delivery systems comprising apomorphine.

More specifically, the invention features compositions and methods for transdermal formulations comprising apomorphine formulated in a patch-needle hybrid (Microneedle) to deliver apomorphine to systemic circulation.

The present invention further includes methods for administering a composition of the present invention to a subject in need thereof. Compositions of the present invention comprising apomorphine can be employed, for example, for the treatment of a variety of conditions and/or disease states which have been historically treated by intravenous and sublingual doses of apomorphine.

The term “subject in need thereof” refers to any animal in need of relief from the symptoms of ED, Alzheimer's, or conditions that can be treated with apomorphine. Preferably, the subject is a mammal. More preferably, the subject is human.

In one embodiment, the present invention provides for a method of treatment for dopaminergic disorders comprising administering apomorphine to a subject having a dopaminergic disorder. In another embodiment, the dopaminergic disorder is selected from the group consisting of Parkinson's disease, juvenile parkinsonism, schizophrenia, depression, drug addiction, Ramsey-Hunt paralysis syndrome.

More particularly, the present invention concerns the transdermal delivery of apomorphine through microneedles. “Apomorphine” refers to the compound: 5,6,6a,7-tetrahydro-6-methyl-4H-dibenzo[de, g]quinoline-10,11-diol, and has the following formula:

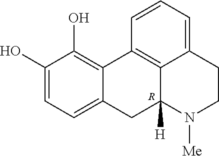

In the present invention, apomorphine can exist in a free base form or as any pharmaceutically acceptable salt. Pharmaceutically acceptable salt refers to pharmaceutically acceptable salts of apomorphine which are derived from a variety of organic and inorganic counter ions that are well known in the art and include, by way of example only, hydrochloride, hydrobromide, tartrate, mesylate, acetate, maleate, oxalate and the like.

For the purposes of the present invention, apomorphine hydrochloride is preferred; however, other pharmacologically acceptable moieties thereof can be utilized as well.

The term “apomorphine” as used herein includes the free base form of this compound as well as pharmacologically acceptable acid addition salts thereof.

The amount of apomorphine administered is an amount sufficient to cause therapeutic effect but is low enough not to cause substantial intolerable adverse side effects. As used herein, “substantial intolerable adverse side effects” include those effects caused by either the delivery system or the active pharmaceutical agent which are incompatible with the health of the user or which are so unpleasant as to discourage the continued use of the composition.

In one embodiment, the pharmaceutical formulation comprises about 0.1 to about 90% wt. apomorphine. In another preferred embodiment a modified release formulation comprises about 1 to about 30% wt. apomorphine.

In one embodiment, the apomorphine is administered in an amount of about 0.1 mg/kg to about 30 mg/kg of body weight. In another embodiment, the apomorphine is administered in an amount of about 0.5 mg/kg to about 20 mg/kg of body weight.

In another embodiment, the pharmaceutical formulation further comprises an additional therapeutic agent useful in treating dopaminergic disorders. In another embodiment, the additional therapeutic agent is selected from the group consisting of levodopa, carbidopa, entacapone, isradapine, bromocriptine, pergolide, pramipexole, ropinirole, benzotropine mesylate, trihexyphenidyl HCl, selegiline, tolcapone, amantadine, riluzole, and L-dopa ethyl ether.

In another embodiment, the pharmaceutical formulation comprises apomorphine and a compound selected from: the group consisting of a testosterone supplement, a PDE V inhibitor, an advanced glycation end-product breaker, an alpha 1 blocker, an alpha 1A antagonist, an alpha 2 antagonism a dopamine agonist, a dopamine D4 agonist, a melanocortin agonist, an oxytocin agonist; a prostaglandin; a radical scavenger; a rotamase inhibitor, a GPCR agonist, a selective androgen receptor modulator (SARM) and a second 5alpha-reductase inhibitor, selected from a compound structural formulae I, II, III or IV.

In another embodiment, the pharmaceutical formulation comprises apomorphine and a compound selected from the group consisting of a testosterone supplement, sildenafil, vardenafil, tadalafil, avanafil, DA159, dasanatafil, SK350, alagebrium chloride, phentolamine mesylate, HMP 12, moxisylyte, yohimbe, spomorphine, NBI69733, ABT724, AT670, BAY632521, PT141; FR229934; SCH444877, ATB901, JNJ10258859 alprostadil, OX008, GPI1485, aviptadil, nitroglycerine, and R873.

In another embodiment, the pharmaceutical formulation comprises apomorphine and a compound selected from the group consisting of a testosterone supplement, levodopa/carbidopa, levodopa/benserazide, ropinirole, apomorphine, selegiline, entacapone, bromocryptine, carbergoline, lysuride, pergolide, orphenadrine, bezhexyl, benztropine and procyclidine, ethopropazine, trihexphenidyl, amitryptaline, doxepine, imipramine, nortriptyline, propanolol, diphenhydramine, orphenadrine, and amantadine.

Pharmaceutical Excipients

The pharmaceutical compositions and drug delivery systems described herein can, if desired, include one or more pharmaceutically acceptable excipients. The term “excipient” herein means any substance, not itself an active pharmaceutical agent, used in conjunction with the active pharmaceutical agent delivered to a subject or added to a pharmaceutical composition or drug delivery system to improve one of more characteristics, such as its handling or storage properties or to permit or facilitate formation of a dose unit of the composition. Excipients include, by way of illustration and not limitation, solvents, thickening agents, penetration enhancers, wetting agents, lubricants, emollients, substances added to mask or counteract a disagreeable odor or flavor, fragrances, and substances added to improve appearance or texture of the composition or drug delivery system. Any such excipients can be used in any dosage forms of the present disclosure. The foregoing list of excipients is not meant to be exhaustive but merely illustrative as a person of ordinary skill in the art would recognize that additional excipients could be utilized.

The compositions and drug delivery systems described herein containing excipients can be prepared by any technique known to a person of ordinary skill in the art of pharmacy, pharmaceutics, drug delivery, pharmacokinetics, medicine or other related discipline that comprises admixing one or more excipients with a therapeutic agent to form a composition, drug delivery system or component thereof.

Non-limiting examples of penetration enhancing agents include sulfoxides such as dimethylsulfoxide and decylmethylsulfoxide; surfactants such as sodium laurate, sodium lauryl sulfate, cetyltrimethylammonium bromide, benzalkonium chloride, poloxamer (231, 182, 184), tween (20, 40, 60, 80) and lecithin; the 1-substituted azacycloheptan-2-ones, particularly 1-n-dodecylcyclazacycloheptan-2-one; fatty alcohols such as lauryl alcohol, myristyl alcohol, oleyl alcohol and the like; fatty acids such as lauric acid, oleic acid and valeric acid; fatty acid esters such as isopropyl myristate, isopropyl palmitate, methylpropionate, and ethyl oleate; polyols and esters thereof such as propylene glycol, ethylene glycol, glycerol, butanediol, polyethylene glycol, and polyethylene glycol monolaurate, amides and other nitrogenous compounds such as urea, dimethylacetamide (DMA), dimethylformamide (DMF), 2-pyrrolidone, 1-methyl-2-pyrrolidone, ethanolamine, diethanolamine and triethanolamine, terpenes; alkanones, and organic acids, particularly salicylic acid and salicylates, citric acid and succinic acid. As noted earlier herein, “Percutaneous Penetration Enhancers”, eds. Smith et al. (CRC Press, 1995), which is incorporated herein by reference thereto, provides an excellent overview of the field and further information concerning possible secondary enhancers for use in conjunction with the present invention. More permeation enhancer(s) suitable to be used with the present invention may be known by those skilled in the art.

The permeation enhancer is present from about 0.1 to about 30% w/w depending on the type of permeation enhancer, a concentration of between about 0.1 and about 10 weight percent, as known by one skilled in the art. In one preferred embodiment, the penetration enhancer comprises myristyl alcohol in a concentration of between about 0.1 and about 2 weight percent.

In another embodiment, the composition comprises antioxidant(s), for example, but not limited to, tocopherol and derivatives, ascorbic acid and derivatives, butylated hydroxyanisole, butylated hydroxytoluene, fumaric acid, malic acid, propyl gallate, and sodium metabisulfite and derivatives

The antioxidant is present from about 0.01 to about 5 weight percent; more preferred embodiment is a concentration of about 0.1 to about 0.5 weight percent, depending on the type of antioxidant used, as known by the one skilled in the art.

In another embodiment, the composition comprises preservatives such as, but not limited to, benzalkonium chloride and derivatives, benzoic acid, benzyl alcohol and derivatives, bronopol, parabens, centrimide, chlorhexidine, cresol and derivatives, imidurea, phenol, phenoxyethanol, phenylethyl alcohol, phenylmercuric salts, thimerosal, sorbic acid and derivatives.

The preservative is present from about 0.01 to about 10% w/w depending on the type of compound used, as known by the one skilled in the art.

Compositions described herein optionally compromise one or more emulsifying agents. The term “emulsifying agent” refers to an agent capable of lowering surface tension between a non-polar and polar phase and includes compounds defined as “self-emulsifying” agents. Suitable emulsifying agents can come from any class of pharmaceutically acceptable emulsifying agents including, but not limited to, carbohydrates, proteins, high molecular weight alcohols, wetting agents, waxes and finely divided solids.

The optional emulsifying agent, if present, is present in a composition in a total amount of about 1% to about 15%, more preferred embodiment is a concentration of about 0.5% to 5%.

In another embodiment, the water immiscible solvent comprises propylene glycol, and is present in a composition in an amount of about 1% to about 99%, by weight of the composition, more preferred embodiment is a concentration of about 1% to about 70% by weight of the composition.

Non-limiting examples of thickening agents include, include hydroxyalkylcelluloses and carboxyalkylcelluloses, such as, hydroxypropylcellulose (HPC), hydroxypropylmethylcellulose (HPMC), hydroxyethylcellulose (HEC), hydroxypropylethylcellulose (HPEC), methyl cellulose (MC), ethyl cellulose (EC), cellulose acetate (CA), cellulose acetate butyrate, cellulose acetate propionate, hydroxypropylmethylcellulose phthalate (HPMCP) (which is also an anionic polymer), carboxyl methylcellulose (CMC), cellulose acetate phthalate (CAP) (which is also an anionic polymer). Examples of pharmaceutically acceptable biologically derived materials include, but are not limited to, polysaccharides or their derivatives, such as, but not limited to, gums (such as, xantham gum, locust bean gum), sodium alginate, shellac, zein, and the like. It might also include anionic polymers such as polyacrylic acid, carboxypolymethylene, carboxymethylcellulose and the like, including derivatives of Carbopol. Polymers. Additional thickening agents, enhancers and adjuvants may generally be found in Remington's The Science and Practice of Pharmacy as well as the Handbook f Pharmaceutical Excipients, Arthur H. Kibbe ed. 2000.

Thickening agents or gelling agents are present in an amount sufficient to provide the desired rheological properties of the composition. Illustratively, one or more pharmaceutically acceptable thickening agent or gelling agent are present in a total amount by weight of about 0.1% to about 20%, more preferred embodiment is a concentration of about 1% to about 10% by weight of the composition.

In one embodiment a neutralizing agent is optionally present to assist in forming a gel. Suitable neutralizing agents include sodium hydroxide (e.g., as an aqueous mixture), potassium hydroxide (e.g., as an aqueous mixture), ammonium hydroxide (e.g., as an aqueous mixture), triethanolamine, tromethamine (2-amino 2-hydroxymethyl-1,3 propanediol), aminomethyl propanol (AMP), tetrahydroxypropyl ethylene diamine, diisopropanolamine, Ethomeen C-25 (Armac Industrial Division), Di-2 (ethylhexyl) amine (BASF-Wyandotte Corp., Intermediate Chemicals Division), triamylamine, Jeffamine D-1000 (Jefferson Chemical Co.), b-Dimethylaminopropionitrite (American Cyanamid Co.), Armeen CD (Armac Industrial Division), Alamine 7D (Henkel Corporation), dodecylamine and morpholine.

The neutralizing agent is present in an amount sufficient to form a gel which is suitable for contact with the skin of a mammal.

In some embodiments according to the present invention, the carrier or vehicle includes one or more solvents, such as C2-C10 alcohols, such as hexanol, cyclohexanol, benzyl alcohol, 1,2-butanediol, glycerol, and amyl alcohol; C5-C10 hydrocarbons such as n-hexane, cyclohexane, and ethylbenzene; C4-C10 aldehydes and ketones, such as heptylaldehyde, cyclohexanone, and benzylaldehyde; C4-C10 esters, such as amyl acetate and benzyl propionate; ethereal oils, such as oil of eucalyptus, oil of rue, cumin oil, limonene, thymol, and 1-pinene; halogenated hydrocarbons having 2-8 carbon atoms, such as 1-chlorohexane, 1-bromohexane, and chlorocyclohexane. Suitable solvents are set forth in U.S. Pat. No. 3,598,122, which is expressly incorporated herein by reference.

Examples of oils comprise fats and oils such as olive oil and hydrogenated oils; waxes such as beeswax and lanolin; hydrocarbons such as liquid paraffin, ceresin, and squalane; fatty acids such as stearic acid and oleic acid; alcohols such as cetyl alcohol, stearyl alcohol, lanolin alcohol, and hexadecanol; and esters such as isopropyl myristate, isopropyl palmitate and butyl stearate. As examples of surfactants there may be cited anionic surfactants such as sodium stearate, sodium cetyl sulfate, polyoxyethylene laurylether phosphate, sodium N-acyl glutamate; cationic surfactants such as stearyldimethylbenzylammonium chloride and stearyltrimethylammonium chloride; ampholytic surfactants such as alkylaminoethylglycine hydrochloride solutions and lecithin; and nonionic surfactants such as glycerin mono stearate, sorbitan monostearate, sucrose fatty acid esters, propylene glycol monostearate, polyoxyethylene oleylether, polyethylene glycol monostearate, polyoxyethylene sorbitan monopalmitate, polyoxyethylene coconut fatty acid monoethanolamide, polyoxypropylene glycol (e.g., the materials sold under the trademark “Pluronic”), polyoxyethylene castor oil, and polyoxyethylene lanolin. Examples of humectants include glycerin, 1,3-butylene glycol, and propylene glycol; examples of lower alcohols include ethanol and isopropanol; examples of thickening agents include xanthan gum, hydroxypropyl cellulose, hydroxypropyl methyl cellulose, polyethylene glycol and sodium carboxymethyl cellulose; examples of antioxidants comprise butylated hydroxytoluene, butylated hydroxyanisole, propyl gallate, citric acid and ethoxyquin; examples of chelating agents include disodium edetate and ethanehydroxy diphosphate; examples of buffers comprise citric acid, sodium citrate, boric acid, borax, and disodium hydrogen phosphate; and examples of preservatives are methyl parahydroxybenzoate, ethyl parahydroxybenzoate, dehydroacetic acid, salicylic acid and benzoic acid. The reservoir may be a void, or may include one or more layers of a suitable material for physically stabilizing the compositions according to the present invention. Suitable materials for the reservoir layer include, for example, polysiloxanes, polyisobutylenes, polyurethanes, plasticized ethylenevinyl acetate copolymers, low molecular weight polyether amide block polymers (e.g., PEBAX), tacky rubbers, such as polyisobutene, polystyrene-isoprene copolymers, polystyrene-butadiene copolymers, and mixtures thereof. The reservoir layer may comprise adhesive materials such as polyisobutylenes, silicones, polyurethanes, and polyacrylates, with polyisobutylenes particularly preferred.

In certain embodiments of the invention, the carrier is composed of the foregoing materials to achieve a controlled occlusion of the skin, thereby resulting in optimal enhancement of biologically active moiety penetration across the skin with minimal skin irritation. In certain embodiments, the reservoir matrix may include a dispersing agent that aids in maintaining a particulate phase comprising the apomorphines dispersed in the continuous phase. In other embodiments, non-ionic excipients, such as lauric alcohol, propylene glycol monolaurate, myristyl lactate, lauryl lactate, or the like, facilitate dispersion.

The rate of biologically active moiety delivery across a dermal surface can be increased by transdermal delivery enhancers. Suitable transdermal delivery enhancers include proton-accepting solvents such as dimethylsulfoxide and dimethylacetamide. Other suitable transdermal delivery enhancers include 2-pyrrolidine, N,N-diethyl-m-toluamide (Deet), 1-dodecylazacycloheptan-2-one (Azone), N,N-dimethylformamide, N-methyl-2-pyrrolidine, terpenes, surfactants, and calcium thioglycolate. However, difficulties remain with such dermal enhancers because the problem of irritation at the site of application has not been overcome.

In some embodiments according to the present invention, the reservoir also includes a hydrogel. Suitable hydrogels for use in a patch according to the present invention include those well known in the art, such as soluble cellulose ethers, e.g., methylcellulose and cellulose derivatives. Other suitable hydrogel materials include blends of either N-vinyl lactam or a copolymer of N-vinyl lactam, an aqueous mixture of a radiation crosslinkable water-soluble polymer such as a polymer of N-vinyl-2-pyrrolidone and ethylene oxide, and a humectant, such as propylene glycol which may be used in transdermal drug delivery system. Suitable hydrogels may contain preservatives such as propyl paraben and methyl paraben.

Suitable materials for the permeable skin contact layer include macroporous rate-controlling materials such as polyvinylchlorides, polyamides, methacrylic copolymers, polysulfones, halogenated polymers, polychloroethers, acetal polymers, acrylic resins, polyurethanes, polyimides, polybenzimidazoles, polyvinylacetate, aromatic, and aliphatic polyethers, cellulose esters, cellulose triacetate, cellulose, cellulose nitrate, epoxy resins, and olefins, such as polyethylenes and polypropylenes.

Dosage forms for the topical or transdermal administration of an apomorphine of this invention include powders, sprays, ointments, pastes, creams, lotions, gels, solutions, patches and inhalants. The apomorphine may be mixed under sterile conditions with a pharmaceutically acceptable carrier, and with any preservatives, buffers, or propellants which may be required.

In a further embodiment, the formulation is a gel, an ointment, a cream or a patch and comprises a pharmaceutically active agent, optionally one or more penetration enhancing agent, thickening agent, lower alcohol, such as ethanol or isopropanol; or water. In another embodiment, the formulation is a gel, an ointment, a cream or a patch, further comprised of sodium hydroxide or triethanolamine or potassium hydroxide, or a combination thereof, in an amount sufficient, as is known in the art, to assist the gelling agent in forming a gel suitable for contact with the skin of a mammal.

In one embodiment, a composition comprises a preservative. Illustrative anti-microbial preservatives include but not limited to benzalkonium chloride and derivatives, benzoic acid, benzyl alcohol and derivatives, bronopol, parabens, centrimide, chlorhexidine, cresol and derivatives, imidurea, phenol, phenoxyethanol, phenylethyl alcohol, phenylmercuric salts, thimerosal, sorbic acid and derivatives.

The preservative is present from about 0.01 to about 10% w/w depending on the type of compound used, as known by the one skilled in the art. Preferred embodiment is from about 1% to about 5%

Compositions described herein optionally comprise one or more pharmaceutically acceptable wetting agents as excipients. Non-limiting examples of surfactants that can be used as wetting agents in compositions of the disclosure include quaternary ammonium compounds, for example benzalkonium chloride, benzethonium chloride and cetylpyridinium chloride, dioctyl sodium sulfosuccinate, polyoxyethylene alkylphenyl ethers, for example nonoxynol 9, nonoxynol 10, and octoxynol 9, poloxamers (polyoxyethylene and polyoxypropylene block copolymers), polyoxyethylene fatty acid glycerides and oils, for example diglycerides. polyoxyethylene sorbitan esters, for example polysorbate 20 and polysorbate 80

The wetting agent, if present, constitute in total from about 0.1% to about 20%, in preferred embodiment from about 1% to about 5%.

Microneedle Array

The pharmaceutical compositions described herein are suitable for use in conjunction with microneedles for transdermal drug delivery which create micrometer-scale transport pathways. Microneedles provide a minimally invasive means to transport molecules into and/or through the skin for local or systemic delivery of an active pharmaceutical agent. The channels or pores created by a microneedle array are extremely small on a clinical level. However, because the channels or pores are orders of magnitude larger than even macromolecules, such channels or pores have been shown to significantly increase skin permeability.

Currently, microneedles are made from silicon, biodegradable polymers, and stainless steel. Microneedles can be solid or hollow. Solid microneedles can be used to create holes in the skin, followed by application of a transdermal patch to the skin surface. Alternatively, solid microneedles can be first coated with a drug and then inserted into the skin. Hollow microneedles can also be used, to facilitate active fluid flow through the needle bore and into the skin. See, e.g., Prausnitz, Adv. Drug. Deliv. Rev. 56 (2004) 581-587, for a review.

The microneedles could be made from silicon, biodegradable polymers, and stainless steel. Microneedles can be solid or hollow. Solid microneedles can be used to create holes in the skin, followed by application of a transdermal patch to the skin surface. Alternatively, solid microneedles can be first coated with a drug and then inserted into the skin. Hollow microneedles can also be used, to facilitate active fluid flow through the needle bore and into the skin. The drug could be formulated in a gel, hydrogel, or any acceptable transdermal formulation to deliver the therapeutic dose.

In one embodiment, the invention provides a method for transdermal delivery of apomorphine, wherein the method comprises the steps of (a) creating a microneedle-treated site in the skin of a subject by inserting microneedles into the skin of said subject, followed by (b) applying apomorphine to the microneedle-treated site. In another embodiment, the invention provides for a microneedle apomorphine-based pharmaceutical agent delivery device having at least one skin-piercing microneedle which comprises a support member coated with a solid reservoir medium containing the apomorphine pharmaceutical agent, and a stabilizing agent that inhibits the degradation effects of free radicals. Alternatively, the skin piercing microneedle may consist of the solid apomorphine pharmaceutical agent reservoir medium without the support member.

On the other hand, the microneedles delivery systems will consist of short, micrometer-scale needles that can be used for drug delivery, allowing a drug to diffuse to the rich capillary bed of the dermis for uptake and subsequent systemic distribution in the blood stream. Since these needles would be inserted no deeper than the outmost, non-innervated layer of the skin, this technique would allow painless delivery. If sufficient bioavailability could be obtained using this route of administration, one could achieve the advantages of subcutaneous drug delivery, but in a non attention drawing and minimally invasive manner. Since studies report needle size and fear of pain as two major reasons for injection anxiety, such a device could improve patient acceptance.

In a preferable embodiment, the apomorphine is administered by creating a microneedle-treated site in the skin of a subject by inserting microneedles, followed by applying the apomorphine to the microneedle-treated site.

In some embodiments according to the present invention, the compositions are applied via a transdermal delivery microneedle device. The microneedles of the device can be constructed from a variety of materials, including metals, ceramics, semiconductors, organics, polymers, and composites. Preferred materials of construction include pharmaceutical grade stainless steel, gold, titanium, nickel, iron, gold, tin, chromium, copper, alloys of these or other metals, silicon, silicon dioxide, and polymers. Examples of microneedle devices that may be used include those set forth in U.S. Publ. Appl. Nos. 20080167601; 20080157427; 20080125743; 20080114298; 20080108959; 20080107720; 20080099963; 20080009825; 20080008745; and 20070276330, and U.S. Pat. Nos. 7,393,517; 7,374,544; 7,344,499; 7,332,197; 7,291,117; 7,262,068; 7,226,439; 7,189,509; 7,132,054; 7,097,776; 7,048,723; 6,980,855; 6,908,453; and 6,611,707, which are all expressly incorporated herein by reference in their entirety for all purposes.

Representative biodegradable polymers include polymers of hydroxy acids such as lactic acid and glycolic acid polylactide, polyglycolide, polylactide-co-glycolide, and copolymers with PEG, polyanhydrides, poly(ortho)esters, polyurethanes, poly(butyric acid), poly(valeric acid), and poly(lactide-co-caprolactone). Representative non-biodegradable polymers include polycarbonate, polymethacrylic acid, ethylenevinyl acetate, polytetrafluorethylene and polyesters.

Generally, the microneedles should have the mechanical strength to remain intact for delivery of biologically active moieties, and to serve as a conduit for the collection of biological fluid and/or tissue, while being inserted into the skin, while remaining in place for up to a number of days, and while being removed. In certain embodiments, the microneedles maybe formed of biodegradable polymers. However, for these embodiments that employ biodegradable materials, the mechanical requirement may be less stringent.

The microneedles can be formed of a porous solid, with or without a sealed coating or exterior portion, or hollow. As used herein, the term “porous” means having pores or voids throughout at least a portion of the microneedle structure, sufficiently large and sufficiently interconnected to permit passage of fluid and/or solid materials through the microneedle. As used herein, the term “hollow” means having one or more substantially annular bores or channels through the interior of the microneedle structure, having a diameter sufficiently large to permit passage of fluid and/or solid materials through the microneedle. The annular bores may extend throughout all or a portion of the needle in the direction of the tip to the base, extending parallel to the direction of the needle or branching or exiting at a side of the needle, as appropriate. A solid or porous microneedle can be hollow. One of skill in the art can select the appropriate porosity and/or bore features required for specific applications. For example, one can adjust the pore size or bore diameter to permit passage of the particular material to be transported through the microneedle device.

The microneedles can have straight or tapered shafts. A hollow microneedle that has a substantially uniform diameter, which needle does not taper to a point, is referred to herein as a “microtube.” As used herein, the term “microneedle” includes, although is not limited to both microtubes and tapered needles unless otherwise indicated. In a preferred embodiment, the diameter of the microneedle is greatest at the base end of the microneedle and tapers to a point at the end distal the base. The microneedle can also be fabricated to have a shaft that includes both a straight (untapered) portion and a tapered portion.

The microneedles can be formed with shafts that have a circular cross-section in the perpendicular, or the cross-section can be non-circular. For example, the cross-section of the microneedle can be polygonal (e.g., star-shaped, square, triangular), oblong, or another shape. The shaft can have one or more bores. The cross-sectional dimensions typically are between about 10 nm and 1 mm, preferably between 1 micron and 200 microns, and more preferably between 10 and 100 μm. The outer diameter is typically between about 10 μm and about 100 μm, and the inner diameter is typically between about 3 μm and about 80 μm.

The length of the microneedles typically is between about 1 μm and 1 mm, preferably between 10 microns and 500 microns, and more preferably between 30 μm and 200 μm. The length is selected for the particular application, accounting for both an inserted and uninserted portion. An array of microneedles can include a mixture of microneedles having, for example, various lengths, outer diameters, inner diameters, cross-sectional shapes, and spacing between the microneedles.

In one embodiment, the microneedle array comprises 15 to 200 microneedles. In one embodiment, the microneedle array comprises 25 to 200 microneedles.

In some embodiments according to the present invention, the apomorphine according to the present invention are encapsulated in a hydrophobic polymer such as polyvinylchloride, optionally plasticized with one or more long-chain fatty acid amides, etc., plasticized nylon, non-plasticized soft nylon, silicone rubber, polyethylene, polyethylene terephthalate; or in a hydrophilic polymer, such as one or more esters of acrylic acid, methacrylic acid, modified collagen, cross-linked hydrophilic polyether gels, cross-linked polyvinylacetate, and cross-linked, partially hydrolyzed polyvinylacetate. Suitable encapsulating agents are set forth in U.S. Pat. No. 3,731,683, which is expressly incorporated herein by reference for all purposes.

The following examples are offered to illustrate this invention and are not to be construed in any way as limiting the scope of this invention.

### EXAMPLES

The following Examples are provided to illustrate certain aspects of the present invention and to aid those of skill in the art in practicing the invention. These Examples are in no way to be considered to limit the scope of the invention in any manner.

### Example 1

### Transdermal Gel Formulation Comprising Apomorphine Hydrochloride

| Ingredient | Percent weight (%) |
|---|---|
| Apomorphine hydrochloride | 3 |
| Hydroxypropylmethylcellulose | 0.05 |
| Methocel E4M | 0.02 |
| Propylene glycol 400 | 15 |
| Ascorbic Acid | 4 |
| Tween 80 | 1 |
| Benzyl alcohol | 1 |
| Ethanol | 10 |
| Water | q.s. |

### Example 2

### Transdermal Delivery of Apomorphine Gel Through Porcine Ear Skin

The skin permeation study was carried out using skin excised from porcine ears. Porcine ears were obtained fresh from a local slaughterhouse and were cleaned under cold running water. The whole skin was removed carefully from the outer region of the ear and separated from the underlying cartilage with a scalpel. Then it was allowed to dry for 30 minutes and afterwards it was wrapped into aluminum foil and stored at −20° C. until use.

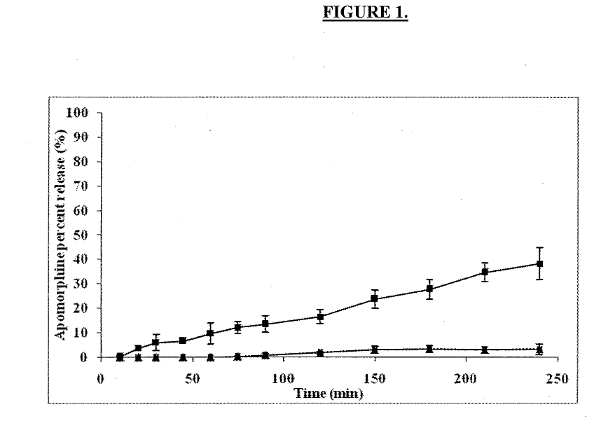

In the experiment day, the skin was taken out of the freezer and thawed. After thawing it was wiped with a cotton ball wetted with phosphate buffer saline (BPS). Skin discs with suitable diameters were mounted onto Franz cells with a nominal area for diffusion of 1.7 cm² and a receptor volume of about 7 mL. The epidermal side was exposed to ambient conditions while the dermal side was bathed with phosphate buffer saline (PBS) pH 7.4 containing 0.01% 2-mercaptoethanol. The receptor fluid was kept at 32±1° C. and constant stirring was maintained by magnetic stirrer at 500 rpm. Care was taken to remove all air bubbles between the underside of the skin (dermis) and the receptor solution throughout the experiment. After conditioning and equilibration for 30 minutes, the apomorphine gel formulation was applied 100 μg/cm² to the skin in the donor compartment of the dissolution cells. The donor compartment was closed securely. Samples were taken from the receptor fluid (200 μL) at predetermined time points and the withdrawn volume was replaced with the same volume of fresh equilibrated PBS buffer to maintain a constant volume. Samples were analyzed by the ultra pressure liquid chromatography mass spectroscopy (UPLC/MS) and the skin permeation data were plotted as the cumulative amount of drug collected in the receiver compartment as a function of time (FIG. 1).

All analytical procedures were performed using a Waters Acquity® Ultra UPLC/MS. An Acquity UPLC BEH Shield RP18 (2.1×100 mm) column (Waters) was used to separate the chemical components. The flow rate was 0.3 mL/min of two mobile phases, (A) made of 5 mM ammonium acetate (pH 4) containing 0.0001% 2-mercaptoethanol and acetonitrile (70:30) and (B) made of acetonitrile. The mass spectrometer was operated in the positive electrospray ionization (ESI) mode. The capillary voltage and cone voltage were maintained at 2.9 kV and 36 V, respectively. The source temperature and desolvation temperature were set at 100 and 350° C., respectively. Mass chromatograms and mass spectral data were acquired and processed by MassLynx software (Waters).

### Example 3

### Transdermal Delivery of Apomorphine Gel Through Porcine Ear Skin Pretreated with Microneedles (150 Micron)

The purpose of this study was to evaluate any enhancement to the transdermal delivery of apomorphine after treatment with microneedles. The skin permeation study was carried out using skin excised from porcine ears as described earlier. After conditioning and equilibration for 30 minutes, the skin samples were removed from the Franz cells and fixed in a Petri dish, the skin samples were perforated with the microneedle patch or the derma rollers with microneedles length of 150 micron. Afterwards the skin samples were mounted back into Franz cell and the apomorphine gel formulation was applied 100 μg/cm² to the skin in the donor compartment of the dissolution cells. The donor compartment was closed securely. Samples were taken from the receptor fluid (200 μL) at predetermined time points and the withdrawn volume was replaced with the same volume of fresh equilibrated PBS buffer containing 0.01% 2-mercaptoethanol to maintain a constant volume. Samples were analyzed by the UPLC/MS analytical method described earlier and the skin permeation data were plotted as the cumulative amount of drug collected in the receiver compartment as a function of time (FIG. 1).

While this invention has been described as having a preferred embodiment, it is understood that the invention is not limited to the illustrated and described features. To the contrary, the invention is capable of further modifications, uses, and/or adaptations following the general principles of the invention and therefore includes such departures from the present disclosure as come within the known or customary practice in the art to which the invention pertains, and as may be applied to the central features set forth above, and which fall within the scope of the appended claims.

It would be obvious to those skilled in the art that modifications or variations may be made to the preferred embodiment described herein without departing from the novel teachings of the present invention. All such modifications and variations are intended to be incorporated herein and within the scope of the claims.

What is claimed is:

1. A transdermal drug delivery system comprising a microneedle delivery system wherein the microneedles of the transdermal delivery system are configured to deliver an active pharmaceutical agent transdermally.

2. The transdermal drug delivery system of claim 1 further comprising an active pharmaceutical agent.

3. The transdermal drug delivery system of claim 2, wherein the active pharmaceutical agent is apomorphine.

4. The transdermal drug delivery system of claim 3, wherein the active pharmaceutical agent is a pharmaceutically acceptable salt of apomorphine.

5. The transdermal drug delivery system of claim 4, wherein the pharmaceutically acceptable salt is apomorphine hydrochloride.

6. The transdermal drug delivery system of claim 3, wherein the active pharmaceutical agent is a prodrug of apomorphine.

7. The transdermal drug delivery system of claim 6, wherein the prodrug is water soluble.

8. The transdermal drug delivery system of claim 3, wherein the microneedles are in the form of a microneedle array.

9. The transdermal drug delivery system of claim 3, wherein the microneedle array comprises 15 to 200 microneedles.

10. The transdermal drug delivery system of claim 3, further comprising a dermal drug delivery patch.

11. The transdermal drug delivery system of claim 3, wherein the active pharmaceutical agent provided in a formulation selected from the group consisting of solutions, gels, nanoemulsions, and nanoparticles formulations.

12. The transdermal drug delivery system of claim 3, wherein the active pharmaceutical agent further comprises a hydrophilic polymer, preservative and an antioxidant.

13. A method for administering apomorphine to a subject in need such treatment, comprising administering a therapeutically effective amount of apomorphine pharmaceutical composition, wherein the apomorphine is administered transdermally using an array of microneedles.

14. The method of claim 13, wherein the apomorphine pharmaceutical composition further comprises a hydrophilic polymer, preservative and an antioxidant.

15. A method of treatment for dopaminergic disorders comprising the steps of (i) contacting the skin with an array of microneedles thereby creating a microneedle-treated site in the skin of a subject, and (ii) applying a therapeutically effective amount of apomorphine to the microneedle-treated site.

16. The method of claim 15, wherein the dopaminergic disorder is selected from the group consisting of Parkinson's disease, juvenile parkinsonism, schizophrenia, depression, drug addiction, Ramsey-Hunt paralysis syndrome and erectile dysfunction (ED).

17. A method for transdermal delivery of apomorphine, wherein the method comprises the steps of: (a) creating a microneedle-treated site in the skin of a subject by inserting microneedles into the skin of the subject, followed by; (b) applying a pharmaceutical composition comprising apomorphine to the microneedle-treated site.

18. The method of claim 17, wherein the microneedle-treated site is created by inserting the microneedles into and removing the microneedles from the skin of the subject prior to application of the apomorphine or any pharmaceutically acceptable salt.

19. The method of claim 17, further comprising the step of abrading a subject's skin.

20. The method of claim 17, wherein the skin is abraded prior to administering the pharmaceutical composition.

21. The method of claim 17, wherein the skin is abraded using a device comprising microneedle array.

22. The method of claim 21, wherein the cross-sectional dimension of the microneedles is between about 1 micron and about 200 microns.

23. The method of claim 21, wherein the outer diameter of the microneedle is from about 10 μm to about 100 μm, and the inner diameter is from about 3 μm to about 80 μm.

24. The method of claim 21, wherein the length of the microneedles typically is from about 1 μm to about 1 mm.

25. The method of claim 21, wherein the microneedle array comprises 15 to 200 microneedles.

26. The method of claim 21, wherein the microneedle array comprises 50 to 100 microneedles.

27. The method of claim 17, wherein the apomorphine is applied to the microneedle-treated site via a topical formulation.

28. The method of claim 17, wherein the topical formulation is selected from the group consisting of a gel, a hydrogel, a topical cream, a salve, and/or an ointment.

29. The method of claim 17, wherein the topical formulation is a gel.

30. The method of claim 17, wherein the pharmaceutical composition comprises about 0.1% to about 90% wt. apomorphine.

31. The method of claim 17, wherein the pharmaceutical composition is a sustained release formulation comprising about 1% to about 30% weight apomorphine.
